(19) United States
(12) Patent Application Publication
(10) Pub. No.: US 2014/0378472 A1
(43) Pub. Date: Dec. 25, 2014

(54) AMORPHOUS VILAZODONE HYDROCHLORIDE, A PROCESS FOR ITS PREPARATION AND PHARMACEUTICAL COMPOSITIONS THEREOF

(71) Applicant: RANBAXY LABORATORIES LIMITED, New Delhi, Delhi (IN)

(72) Inventors: Poonam Kaushik, Gurgaon (IN); Ram Thaimattam, Hyderabad (IN); Mohan Prasad, Gurgaon (IN); Sudershan Kumar Arora, Gurgaon (IN)

(21) Appl. No.: 14/363,393

(22) PCT Filed: Dec. 12, 2012
(86) PCT No.: PCT/IB2012/057247

§ 371 Date: Jun. 6, 2014

(30) Foreign Application Priority Data
Dec. 12, 2011 (IN) 3608/DEL/2011

(51) Int. Cl. A61K 31/496

(52) U.S. Cl. CPC A61K 31/496
USPC 514/254.09; 544/373

(57) ABSTRACT

The present invention relates to amorphous vilazodone hydrochloride, its process of preparation and pharmaceutical composition thereof.

### FIELD OF THE INVENTION

The present invention relates to amorphous vilazodone hydrochloride, its process of preparation, and pharmaceutical compositions thereof.

### BACKGROUND OF THE INVENTION

Vilazodone hydrochloride is chemically described as 5-{4-[4-(5-cyano-1H-indo1-3-yl)butyl]piperazin-1-yl}-1-benzofuran-2-carboxamide hydrochloride of Formula I.

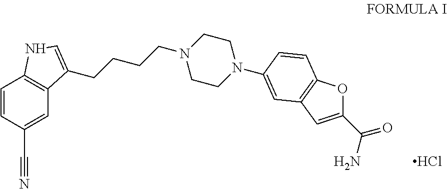 [SMILES: N#Cc1ccc2[nH]cc(CCCCN3CCN(c4ccc5oc(C(N)=O)cc5c4)CC3)c2c1 ; Cl]

Vilazodone hydrochloride is indicated for the treatment of major depressive disorder (MDD).

Processes for the preparation of vilazodone hydrochloride and its various polymorphic forms are described in U.S. Pat. Nos. 5,532,241; 7,834,020; 7,981,894; and 7,381,726; U.S. Publication Nos. 2011/0183994 and 2011/0190317; and European Patent Nos. EP 1 397 357 and EP 0 648 767.

### SUMMARY OF THE INVENTION

The present invention relates to an amorphous vilazodone hydrochloride, its process of preparation, and pharmaceutical compositions thereof.

### BRIEF DESCRIPTION OF THE DRAWINGS

FIG. 1 depicts the X-ray Powder Diffraction Pattern (XRPD) of the amorphous vilazodone hydrochloride obtained according to Example 1.

FIG. 2 depicts the X-ray Powder Diffraction Pattern (XRPD) of the amorphous vilazodone hydrochloride obtained according to Example 2.

FIG. 3 depicts the X-ray Powder Diffraction Pattern (XRPD) of the amorphous vilazodone hydrochloride obtained according to Example 3.

FIG. 4 depicts the X-ray Powder Diffraction Pattern (XRPD) of the amorphous vilazodone hydrochloride obtained according to Example 4.

FIG. 5 depicts the X-Ray Diffraction Pattern (XRPD) of the amorphous vilazodone hydrochloride obtained according to Example 3 after storage at 25° C. and 52% relative humidity (RH) for 24 days.

FIG. 6 depicts the Differential Scanning calorimetry (DSC) of the amorphous vilazodone hydrochloride obtained according to Example 2.

FIG. 7 depicts the Differential Scanning calorimetry (DSC) of the amorphous vilazodone hydrochloride obtained according to Example 3.

### DETAILED DESCRIPTION OF THE INVENTION

A first aspect of the present invention provides an amorphous vilazodone hydrochloride.

The term “amorphous” refers to a solid without long-range crystalline order. The amorphous form of a compound of Formula I of the present invention preferably contains less than about 20% crystalline forms, more preferably less than 5% crystalline forms, and still more preferably less than 1% or is essentially free of crystalline forms. “Essentially free of crystalline forms” means that no crystalline polymorph forms can be detected within the limits of an X-ray Powder Diffractometer.

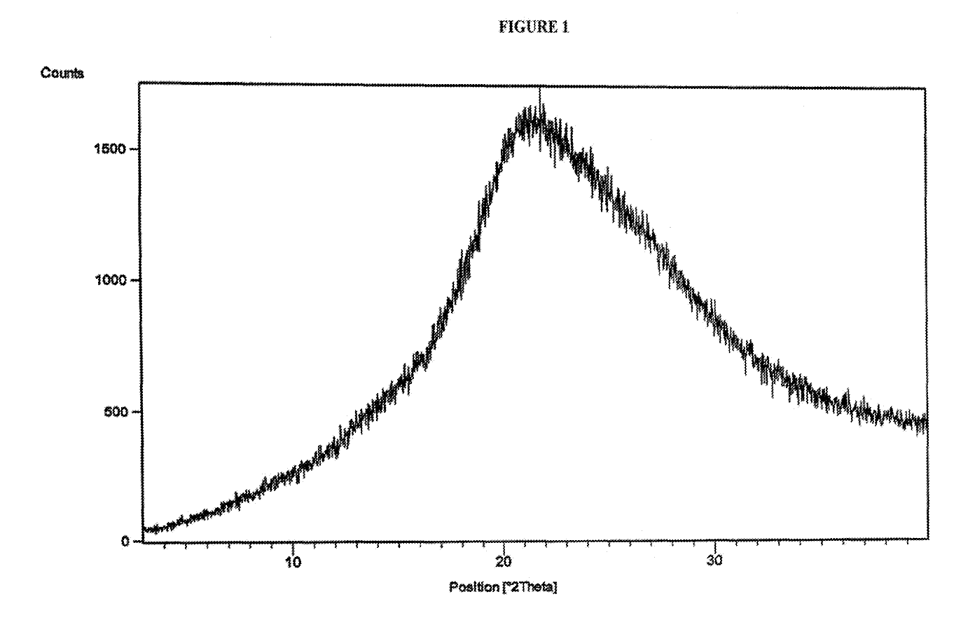

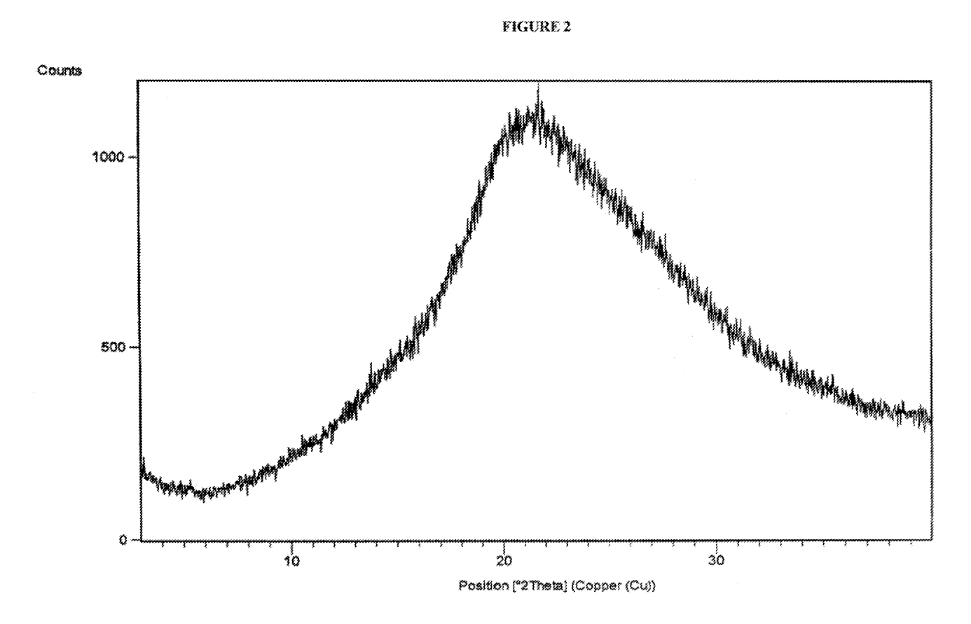

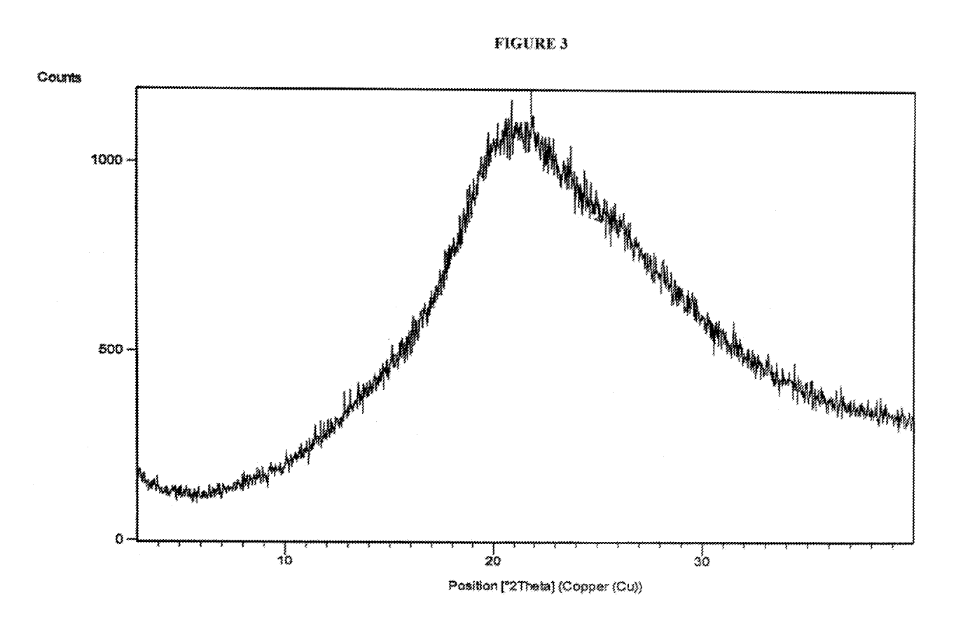

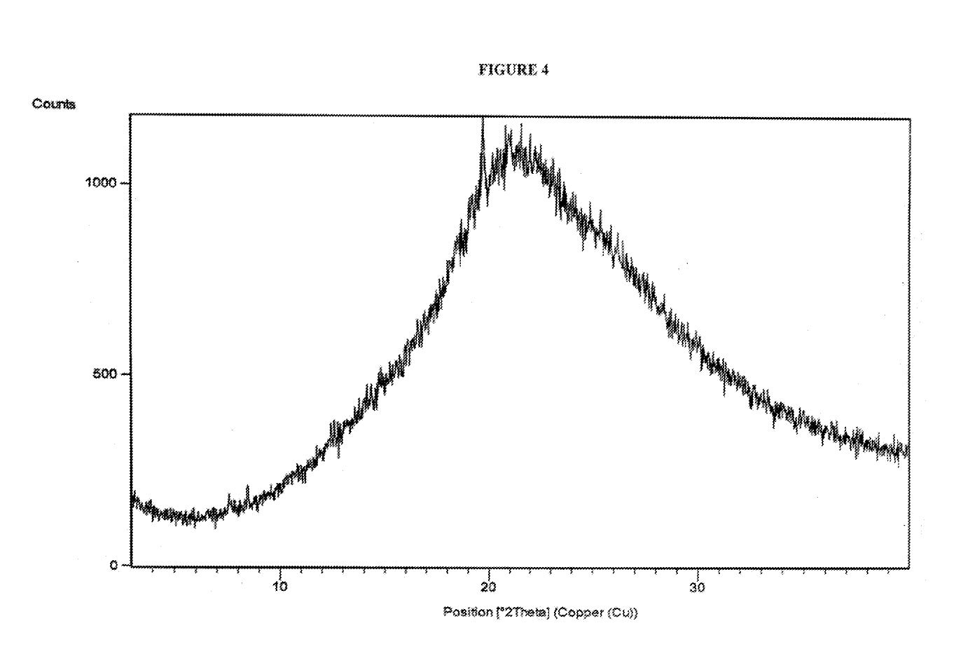

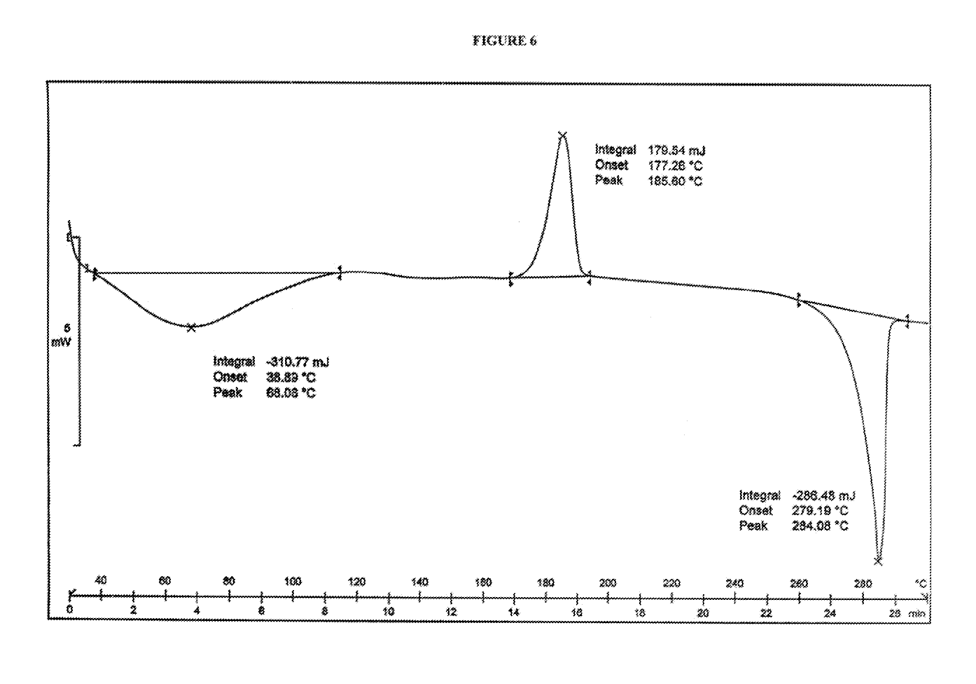

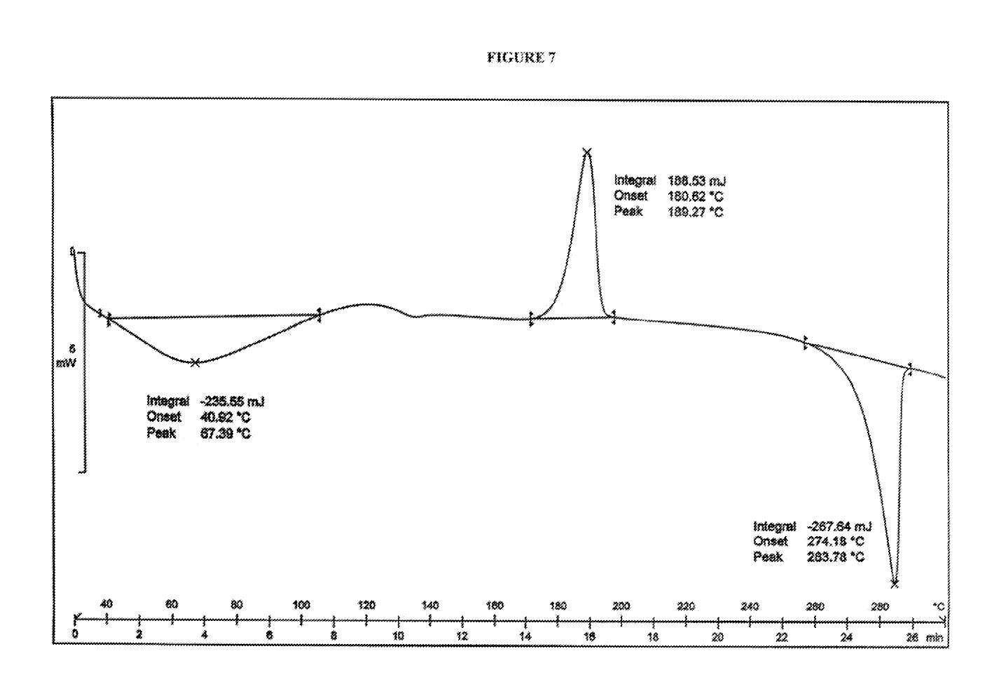

The amorphous vilazodone hydrochloride prepared by the present invention may be characterized by an X-ray Powder Diffraction Pattern (XRPD) as depicted in FIG. 1, FIG. 2, FIG. 3, or FIG. 4. The amorphous vilazodone hydrochloride prepared by the present invention may be further characterized by DSC data as depicted in FIGS. 6 and 7.

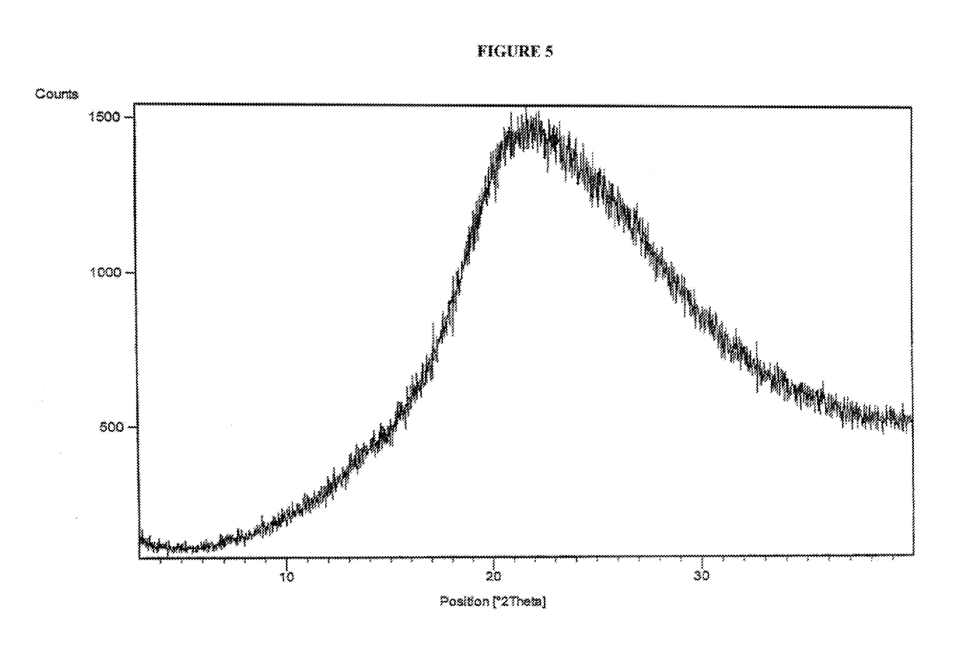

The amorphous vilazodone hydrochloride prepared by the present invention is stable and does not convert to any other polymorphic form on storage at 25° C. and 52% relative humidity (RH) for 24 days as depicted by X-ray Powder Diffraction Pattern (XRPD) pattern similar to FIG. 5.

A second aspect of the present invention provides a process for the preparation of an amorphous vilazodone hydrochloride wherein the process comprises:

a) obtaining a solution of vilazodone hydrochloride;

b) removing the solvent from the solution obtained in step a); and

c) isolating amorphous vilazodone hydrochloride from the reaction mixture.

A solution of vilazodone hydrochloride can be obtained by treating vilazodone hydrochloride with one or more solvents.

The term “solvent” includes any solvent or solvent mixture, for example, water, esters, alkanols, halogenated hydrocarbons, ketones, ethers, polar aprotic solvents, or mixtures thereof.

The solvent may be selected from the group consisting of water, alkanol, esters, ketones, ethers, polar aprotic solvents, or mixtures thereof. Examples of alkanols include those primary, secondary, and tertiary alcohols having from one to six carbon atoms. Suitable alkanol solvents include methanol, ethanol, n-propanol, 2-propanol, and butanol. Examples of ester solvents include ethyl acetate, n-propyl acetate, isopropyl acetate, and n-butyl acetate. Examples of ketones include acetone, methyl ethyl ketone, and the like. Examples of ethers include tetrahydrofuran and the like. A suitable polar aprotic solvent includes N,N-dimethylformamide, N,N-dimethylacetamide, dimethylsulphoxide, acetonitrile, and N-methylpyrrolidone. Examples of halogenated hydrocarbons include dichloromethane, chloroform, and 1,2-dichloroethane. A solvent may preferably be a mixture of water with alkanol, for example, a mixture of water with methanol, ethanol, or 2-propanol.

Treating vilazodone hydrochloride with one or more solvents may include adding, dissolving, slurrying, stirring, or a combination thereof. Vilazodone hydrochloride may be treated with a solvent at a temperature of about 60° C. to about 100° C., preferably at about 70° C. to about 80° C.

The solvent may be removed in step b) by using various drying techniques, for example, spray drying, vacuum drying, freeze drying, or agitated thin film drying.

Isolation of the amorphous vilazodone hydrochloride in step c) comprises a common isolation technique such as evaporation, evaporation under vacuum, cooling, extraction, one or more washings, crystallization, precipitation, filtration, filtration under a vacuum, decantation and centrifugation, or a combination thereof.

A third aspect of the present invention provides a pharmaceutical composition comprising an amorphous vilazodone hydrochloride and a carrier.

A fourth aspect of the present invention provides a method of treating or preventing major depressive disorder (MDD) comprising a step of administering to a patient in need thereof of a therapeutically effective amount of amorphous vilazodone hydrochloride.

XRPD of the samples were determined by using a Panalytical X'Pert Pro X-Ray Powder Diffractometer in the range 3-40 degree 2 theta and under a tube voltage and current of 45 Kv and 40 mA, respectively. Copper radiation of wavelength 1.54 angstrom and Xceletor detector was used.

In the following section, embodiments are described by way of examples to illustrate the process of invention. Several variants of these examples would be evident to persons ordinarily skilled in the art.

### EXAMPLES

### Example 1

### Preparation of Amorphous Vilazodone Hydrochloride

Vilazodone hydrochloride (5.0 g) was dissolved in methanol (125 mL) and water (15 mL) at 80° C. The reaction mixture was filtered and spray dried under the following conditions:

Air Inlet Temperature: 100° C.

Air Outlet Temperature: 52° C.

The solid so obtained was collected from the spray dryer and dried in a vacuum tray drier at 55° C. for 4 hours to obtain the title compound having an XRPD pattern as depicted in FIG. 1.

Yield: 1.52 g

### Example 2

### Preparation of Amorphous Vilazodone Hydrochloride

Vilazodone hydrochloride (2.5 g) was dissolved in 2-propanol (125 mL) and water (125 mL) at 80° C. The reaction mixture was filtered and spray dried under the following conditions:

Air Inlet Temperature: 130° C.

Air Outlet Temperature: 66° C.

The solid so obtained was collected from the spray dryer and dried in a vacuum tray drier at 55° C. for 4 hours to obtain the title compound having an XRPD pattern as depicted in FIG. 2.

Yield: 2.08 g

### Example 3

### Preparation of Amorphous Vilazodone Hydrochloride

Vilazodone hydrochloride (5.12 g) was dissolved in ethanol (125 mL) and water (125 mL) at 71° C. The reaction mixture was filtered and spray dried under the following conditions:

Air Inlet Temperature: 120° C.

Air Outlet temperature: 65° C.

The solid so obtained was collected from the spray dryer and dried in a vacuum tray drier at 55° C. for 4 hours to obtain the title compound having an XRPD pattern as depicted in FIG. 3.

Yield: 2.32 g

### Example 4

### Preparation of Amorphous Vilazodone Hydrochloride

Vilazodone hydrochloride (0.6 g) was dissolved in methanol (60 mL) and water (5 mL) at 70° C. The solvent was quickly distilled on a Buchi Rotovapor under the following conditions:

Temperature: 70° C.

Rotations per minute: 200

Pressure: 55 mbar

The solid so obtained was collected from the Buchi Rotovapor and dried in a vacuum tray drier at 55° C. for 4 hours to obtain the title compound having an XRPD pattern as depicted in FIG. 4.

Yield: 0.46 g

1. Amorphous vilazodone hydrochloride.

2. Amorphous vilazodone hydrochloride according to claim 1 which is characterized by X-ray powder diffraction pattern (XRPD) pattern as depicted in FIG. 1, 2, 3, or 4.

3. Amorphous vilazodone hydrochloride according to claim 1 which is characterized by DSC data as depicted in FIG. 6 or 7.

4. Amorphous vilazodone hydrochloride according to claim 1 which contains less than about 20% crystalline forms.

5. (canceled)

6. Amorphous vilazodone hydrochloride according to claim 1 which is essentially free of crystalline forms.

7. Stable amorphous vilazodone hydrochloride.

8. The stable amorphous vilazodone hydrochloride according to claim 7 which does not convert to any other polymorphic form on storage at 25° C. and 52% relative humidity (RH) for 24 days.

9. The stable amorphous vilazodone hydrochloride according to claim 7 which is characterized by the X-ray Powder Diffraction Pattern (XRPD) pattern as depicted in FIG. 5 on storage at 25° C. and 52% relative humidity (RH) for 24 days.

10. A process for the preparation of amorphous vilazodone hydrochloride wherein the process comprises:
a) obtaining a solution of vilazodone hydrochloride;
b) removing the solvent from the solution obtained in step a); and
c) isolating amorphous vilazodone hydrochloride from the reaction mixture.

11. The process according to claim 10, wherein the solution of vilazodone hydrochloride is obtained by treating the vilazodone hydrochloride with one or more solvents.

12. The process according to claim 11, wherein the solvent is selected from the group consisting of water, alkanol, esters, ketones, ethers, polar aprotic solvents, or mixtures thereof.

13-17. (canceled)

18. The process according to claim 11, wherein the solvent is a mixture of water with methanol, ethanol, or 2-propanol.

19. The process according to claim 10, wherein the solvent is removed in step b) by using drying techniques.

20. (canceled)

21. A pharmaceutical composition comprising an amorphous vilazodone hydrochloride and a carrier.

22. A method of treating or preventing a major depressive disorder (MDD) comprising a step of administering to a patient in need thereof of a therapeutically effective amount of an amorphous vilazodone hydrochloride.
